(19) United States
(12) Patent Application Publication
(10) Pub. No.: US 2005/0061333 A1
(43) Pub. Date: Mar. 24, 2005

(54) Dermawear

(76) Inventor: Reginald Laral Hart, Chicago, IL (US)

Correspondence Address: Reginald Hart, P. O. BOX 806551, CHICAGO, IL, 60680-4126, US

(21) Appl. No.: 10/667,634

(22) Filed: Sep. 22, 2003

(51) Int. Cl. A61F 13/00

(52) U.S. Cl. 128/891

(57) ABSTRACT

Apart from concrete existence the theoretical concept of my intellectual property is one of defense and/or protection. In the abstract, the spiritual (not of flesh or material matter) force drawn upon, and/or drawn out from my inspired (of God's breath) mind of invention (imagination) is, the principles of the Most High's “Grace and Mercy” towards fornicators and adulterers. “Grace and Mercy” manifested as “Dermawear” (Derm-a-Wear): Derma equals skin; wear equals an act or manner of covering—hence, protection. Protective skin wear against sexually transmitted diseases of the male epidermic pubic and scrotal region.

### BACKGROUND OF THE INVENTION

“Dermawear” residing in the medical field is a subgarment of cloth equipment set forth as a useful stratagem of skill and ingenuity in defeat of, or against the inoculation of sexually transmitted diseases of the male genital region.

Endeavoring to achieve a purposeful end of health for all men, “Dermawear” is classified as medical equipment, because the related subject matter of sexually transmitted diseases in this invention (Dermawear), on behalf of the male genital region, warrants it and further defines the scope of “Dermawear's” functioning authority to thwart the attainment of skin pathogens upon the male pubic area.

Prior Art

“Not Applicable.”

### BRIEF SUMMARY OF THE INVENTION

The substance of “Dermawear” is its practical use as a protective subgarment (Fabric Shield) during sexual intercourse.

The utility object of “Dermawear” is its specific action of purpose to protect its user from sexually transmitted diseases, since prophylactic use alone leaves the pubic hairs and scrotal skin exposed during genital copulation; “Dermawear” protects these exposed areas.

### BRIEF DESCRIPTION OF THE DRAWINGS

FIG. 1A depicts a front view of “Dermawear.” “Dermawear”: (Fabric Shield).

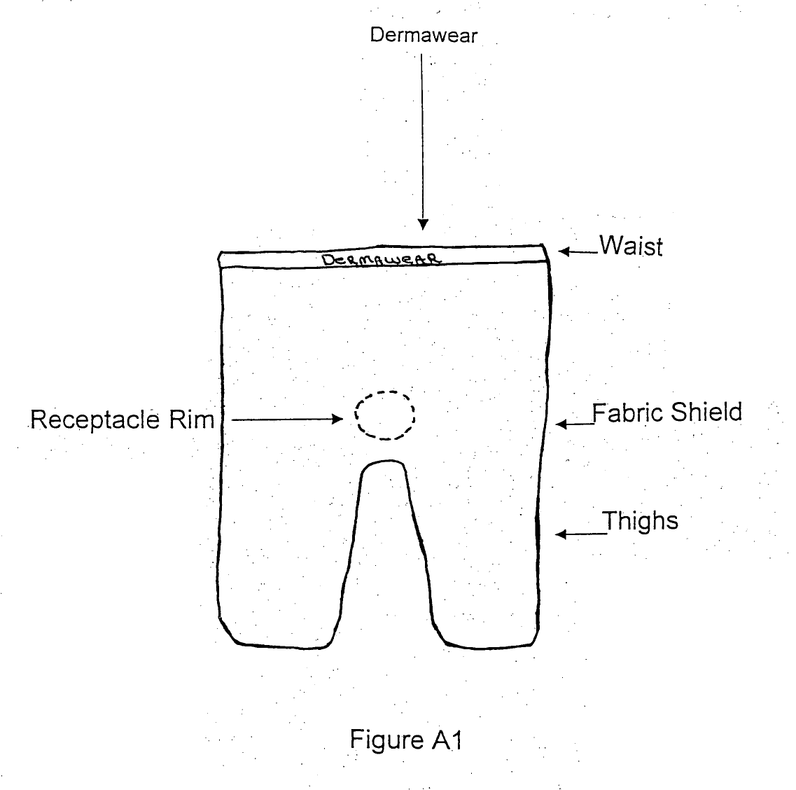

FIG. 2B is an arrow pointing at the “Receptacle Rim.” “Dermawear”: (Receptacle Rim).

FIG. 3C is a representation of the different borders that the “Receptacle Rim” can have as a shape.

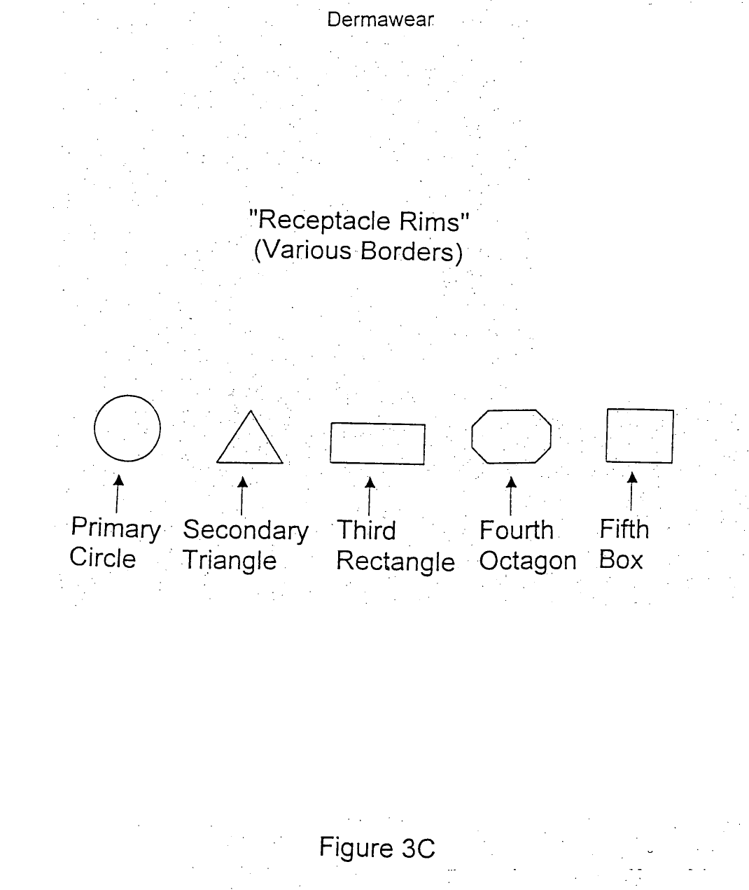

### DETAILED DESCRIPTION OF THE INVENTION

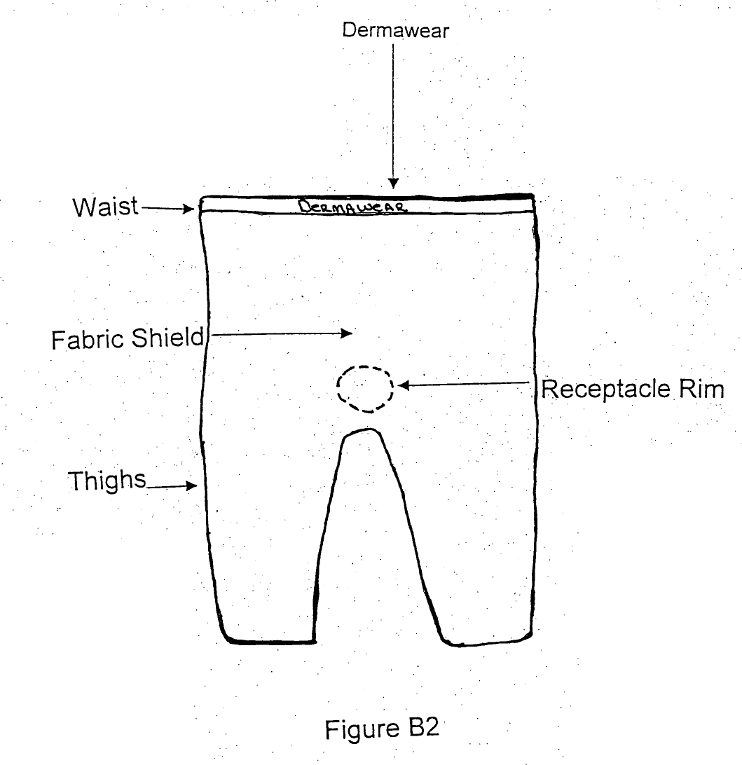

To produce “Dermawear,” a person of ordinary skill in the pertinent art of subgarment manufacturing can make this invention without experimentation, by simply taking 100 percent cotton fabric and interlocking (weaving tightly) the material and double butting (a 2 in 1 subgarment) the cloth; and in the center of the subwear at exactly the location of the penis, cut out a 1/2 inch hole (See FIG. 2B/Drawings) or “Receptacle Rim.” The hole (“Receptacle Rim”) may be set forth in a circle (or triangle, rectangle, octagon) as its primary border.

“Dermawear” is set apart as a new invention, predicated solely upon its intent or application of utility (use and/or function). That is, “Dermawear” and the “Receptacle Rim” is newly functional based upon the intent of use by its user.

“Dermawear's” intent of application makes it dissimilar from all other subgarments, and therefore it is a new invention.

### DETAILED DESCRIPTION OF THE INVENTION

The “Receptacle Rim,” (1) Receptacle: a container, as in the subgarment itself, containing the male genital region; (2) Rim: a hole within the garment primarily bordered as a half inch circle which serves as an outlet for what is contained; in the case of “Dermawear,” that would be an erect penis.

“Dermawear” as a subsgarment with its “Receptacle Rim” is a combined function of use (utility): protective (fabric shield) and an outlet (Receptacle Rim): (1) protective of pubic hair and scrotal skin; (2) and a safe outlet for an erect prophylactic penile shaft.

Independent claim #1:

1. In chattel, what I claim as my invention is: the “Receptacle Rim” of “Dermawear,” which cannot be independent of the subgarment (fabric shield) since the subgarment itself serves as the protective aspect of “Dermawear”; and the “Receptacle Rim” is an outlet for an erect penis.
Independent claim #1:
What I claim as my invention is: the subgarment made of any type of fabric, but primarily consisting of 100 percent cotton set forth and combined with a “Receptacle Rim,” which are dependent components in alliance for one purpose: to protect the male pubic region and scrotal skin from sexually transmitted diseases; and in the nature of this purpose exists “Dermawear,” my claim of invention.
Independent claim #1:
“Dermawear” is a pioneering concept, and what I claim as my invention is the “Receptacle Rim” and its shielding subgarment made of any type of material (polyester, rayon, tweed, etc.), but primarily consisting of 100 percent cotton.
Independent claim #1:
What I claim as my invention is a subgarment (fabric shield) with its “Receptacle Rim” worn only at the time of sexual copulation, and therefore distinguished by its consumer purchase for intent of application; and the intent of application is established within the intent of manufacture of “Dermawear” (fabric shield) and its “Receptacle Rim.” A claim “within intent of manufacture” means applied use or intent of application by consumers for the sole purpose of protecting themselves against sexually transmitted diseases; hence, “Dermawear,” my claim of invention.
Independent claim #2:
What I claim as my invention is “Dermawear's” intent of application: “Dearmawear” is not for the vanity of fashion as an intent of consumer application with respect to appearance, but it is protective wear.
Independent claim #3:
What I claim as my invention is “Dermawear's” intent of application: “Dermawear” is not for the ordinary and common consumer use as a subgarment of comfort against the friction of surface apparel, but it is protective wear against sexually transmitted diseases.
Dependent upon claim #1:
What I claim as my invention is: the nonobvious distinction of “Dearmawear” as a tactical use against diseases of the skin-contrasting and/or set apart from the practical ordinary and obvious use of subgarments worn with a consumer intent of application (applied use) for comfort alone.
Dependent upon claim #1:
What I claim as my invention is: “Dermawear's” intent of application (applied use) as a medical device of tactical equipment wear. Tactical: characterized by skill of use and distinct from the ordinary use of subgarments for comfort alone.
Dependent upon claim #1:
What I claim as my invention is: “Dermawear's” intent of manufacture for tactical consumer use against sexually transmitted diseases; so that, I claim the utility of “Dermawear” in chattel: a “fabric shield” and a “Receptacle Rim,” is my intellectual property of invention.
Dependent upon claim #1:
What I claim as my invention is” “Dermawear” or subgarment manufacturing to be sold in the subgarment market place for the sole purpose and/or intent of application by consumers protecting themselves with an article of manufactured medical equipment wear against sexually transmitted disease of the pubic region and scrotal skin.
Dependent upon claim #1:
What I claim as my invention is: “Dermawear” as a new and useful process of protection during the act or method of sexual intercourse against the acquisition of sexually transmitted diseases by skin-to-skin contact.
Dependent upon claim #1:
What I claim as my invention is” “Dermawear” juxtaposed: ordinary and common subgarments for men are primarily used as a function of comfort against the friction of surface apparel (namely pants/shorts); and ancillary use for ordinary and common subgarments serve as a function of retention against sudden and unexpected bowel movements of human excrement: urine and fecal matter. Ordinary and common (obvious) subgarments for male use, contain in front an elongated vertical slit, for easy access to the penis, for the consumer intent of application as a biological function of urination.
Dependent upon claim #1:
What I claim as my invention is: “Dermawear” juxtaposed: some ordinary and common subgarments for men do not contain in front an elongated slit at all; but require men to pull the subgarment midway down the thigh for excretory urinary and/or fecal function.
Dependent upon claim #2:
What I claim as my invention is: “Dermawear” juxtaposed: ordinary and common subgarments for men are made of overly porous fabric material; and when an elongated slit is present, its intent of application (applied use) is for the excretory urinary function of the penis, which offers no process (tightly fitted 1/2 inch “Receptacle Rim”) of protection from or against a sexually transmitted disease.
Dependent upon claim #2:
What I claim as my invention is” “Dermawear” juxtaposed: comparatively, “Dermawear” is nonobvious in that it is made of interlocking and tightly woven, double butted (2 in 1 subgarment) 100 percent cotton material, and has a protective function and uses a half-inch circle (or triangle, or octagon, or rectangle) or “Receptacle Rim” invented to fit tightly against and/or around the base of the penis.
Dependent upon claim #2:
What I claim as my invention is: “Dermawear” juxtaposed: a subgarment or “fabric shield” with a “Receptacle Rim” and an intent of manufacture for consumer use against sexually transmitted diseases.
Dependent upon claim #2:
What I claim as my invention is: the legal merit of “Dermawear” or protective subgarment—and its nonobvious use as safe skin wear against sexually transmitted diseases; so as to be nonobvious to a person having ordinary skill in the medical equipment area of S.T.D. technology.
Dependent upon claim #2:
What I claim as my invention is: the dejure of “Dermawear” as a subgarment for medical use (intent of application) and a preventive classification, expressed as a “medium (barrier) process” of protection from sexually transmitted diseases of the male genital area (pubic region and scrotal skin).
Dependent upon claim #2:
What I claim as my invention is: “Dermawear”—and its intent of application through its new and specific attribute of a “Receptacle Rim” set forth as a 1/2 inch circular hole.
Dependent upon claim #2:
What I claim as my invention is: “the utility right of “Dermawear,” or subgarment protective skin wear (P.S.W.) as my intellectual property.
Dependent upon claim #3:
What I claim as my invention is: “Dermawear's” Technical Merit of Protection against: (1) Genital Human Papillomavirus (2) Sarcoptes Scabiei (Scabies); (3) Phthirus Pubis (Crabs); (4) Cancerous Parasitic Mutated Mite (C.P.M.M./Protein fusion of skin).
Dependent upon claim #3:
What I claim as my invention is: “Dermawear” technical merit of protection against: (1) Biofluid Hazards; (2) Viral shedding; (3) Purulent material from lesions (Biofluids); (4) Chancroid; (5) Pruritus of the pubic area; (6) any skin-to-skin contact, as “Dermawear” is a medium barrier between human to human disease inoculation.
Dependent upon claim #3:
Wherever use can be demonstrated then utility has been defined. “Dermawear” is the use of protection against sexually transmitted diseases.
Dependent upon claim #3:
“Dermawear” is the specificity of use, set forth as the intent of application (applied use) against S.T.D.'s.
Dependent upon claim #3:
The functional parts of “Dermawear's” subgarment are: (1) “fabric shield,” and (2) “Receptacle Rim” purposed as the complete entity of utility protection against sexually transmitted diseases.
Dependent upon claim #3:
Claim: (1) Receptacle Rim; (2) Fabric Shield; (3) Intent of manufacture resulting in consumer use of purchase as an intent of application or applied use for protection against sexually transmitted diseases. “Dermawear” is mindful of the female anatomy and stands ready to be manufactured for consumer use by women for protection against sexually transmitted diseases.
